(19) United States
(12) Patent Application Publication
(10) Pub. No.: US 2007/0038030 A1
(43) Pub. Date: Feb. 15, 2007

(54) Endoscope

(75) Inventors: Kunikiyo KANEKO, Saitama (JP); Hiroto WATANABE, Tokyo (JP); Satoshi TAKAMI, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/463,450

(22) Filed: Aug. 9, 2006

(30) Foreign Application Priority Data
Aug. 10, 2005 (JP) 2005-231748

(51) Int. Cl. A61B 1/06

(52) U.S. Cl. 600/180

(57) ABSTRACT

An endoscope has a plurality of LEDs (Light-Emitting Diodes) that illuminate an object and are disposed at a tip portion of the endoscope, and a light-controller that controls an emission of the plurality of LEDs. The light-controller controls the plurality of LEDs so as to cause a luminance difference among the plurality of LEDs.

### BACKGROUND OF THE INVENTION

1. Field of the Invention

The present invention relates to an endoscope that observes an object using light irradiated from a light source. In particular, it relates to an endoscope that has an LED (Light-Emitting Diode) as a light source.

2. Description of the Related Art

In an endoscope with LEDs, light for illuminating an observed portion is irradiated from the LED provided at the distal end of a video-scope or fiber scope. An observed image is formed by the reflected light, and the operator diagnoses or operates while seeing the observed image. For example, an LED emitting white-light is disposed at the distal end of the scope.

When illuminating the observed portion uniformly, the solidity or three-dimensionality of an observed image does not exhibit clearly, which makes it difficult to diagnose a diseased portion. Also, while inserting the endoscope, the tip portion of the endoscope faces a longitudinal direction of the body-cavity; therefore, the inner wall of the body-cavity, which is displayed around the observed image, is close to the tip portion, whereas a hollow portion of the body-cavity, which is displayed at the center area, is distant from the tip portion. Consequently, when illuminating the observed portion uniformly, halation occurs in the surrounding area, and the observed portion to be diagnosed is not clearly displayed.

### SUMMARY OF THE INVENTION

An object of the present invention is to provide an endoscope that is capable of displaying a clear observed image of a body-cavity with solidity.

An endoscope according to the present invention has a plurality of LEDs (Light-Emitting Diodes) that illuminate an object and are disposed at a tip portion of the endoscope, and a light controller that controls the plurality of LEDs so as to cause a luminance difference among the plurality of LEDs.

An endoscope of another aspect according to the present invention has a plurality of LEDs (Light-Emitting Diodes) that illuminate an object and are disposed at a tip portion of the endoscope; and a light controller that controls an emission of the plurality of LEDs, each LED having a different directivity of light. The plurality of LEDs includes LEDs that have different directivities of light.

### BRIEF DESCRIPTION OF THE DRAWINGS

The present invention will be better understood from the description of the preferred embodiments of the invention set forth below together with the accompanying drawings, in which:

FIG. 1 is a block diagram of a fiber scope according to a first embodiment;

FIG. 2 is a block diagram of the light-controller;

FIGS. 3A and 3B are views showing luminance distributions of the LEDs;

FIG. 4 is a block diagram of an electronic endoscope according to a second embodiment;

FIG. 5 is a view showing a front view of the tip portion; and

FIGS. 6A and 6B are views showing directivities of the LEDs.

### DESCRIPTION OF THE PREFERRED EMBODIMENTS

Hereinafter, the preferred embodiments of the present invention are described with reference to the attached drawings.

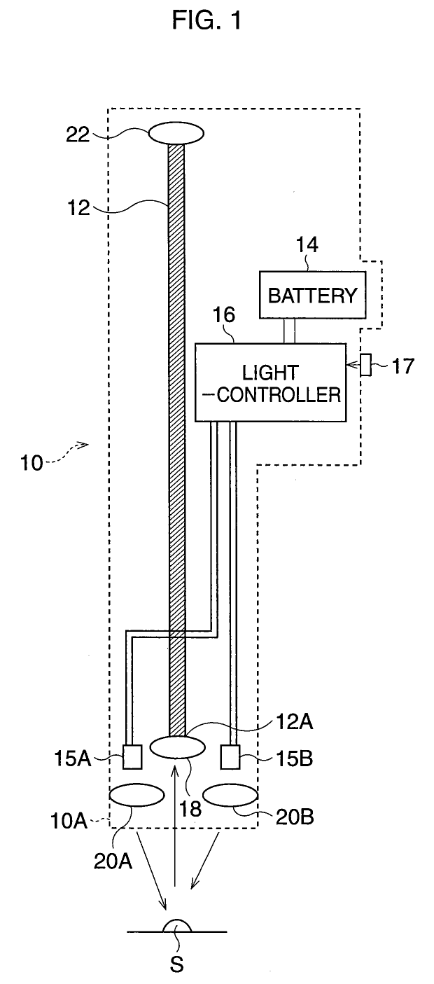

FIG. 1 is a block diagram of a fiber scope according to a first embodiment.

A fiber scope 10 has an image-fiber 12 of a fiber-optic bundle, and has two LEDs 15A and 15B, which are disposed at the tip portion of the fiber scope 10. Each of LEDs 15A and 15B is covered with a resin-lens. A battery 14 supplies electric power to a light-controller 16. The light-controller 16 turns the LEDs 15A and 15B ON in accordance with electric power supply. When the LEDs 15A and 15B are turned ON, light emitted from the LEDs 15A and 15B pass through a diffusion lens (not shown), and is irradiated from the tip portion 10A. Consequently, an observed portion is illuminated by the irradiated light.

Light reflected off the observed portion passes through an objective lens 18, so that an observed image is formed on the tip surface 12A of the image-fiber 12. The image-fiber 12 optically transmits the observed image to the opposite tip surface of the image-fiber 12. The operator watches the observed image via an eyepiece 22. A color balance button 17 is a button for changing a resistor value of a variable resistor (herein, not shown). The operator sets the resistor value by manipulating the color balance button 17. The LEDs 15A and 15B are arranged so as to be symmetrical with respect to the center axis of the tip portion 10A, and are arranged around the objective lens 18 at regular intervals.

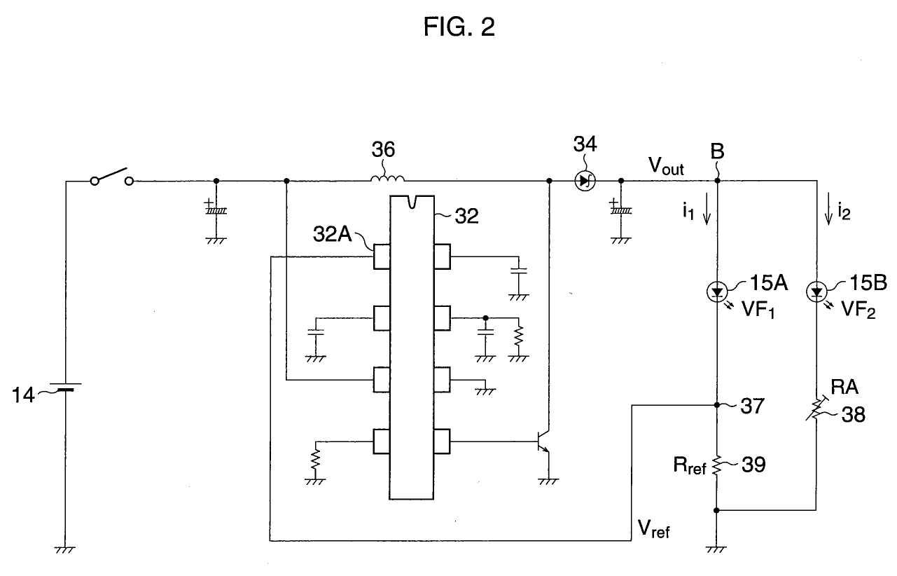

FIG. 2 is a block diagram of the light-controller 16. The light-controller 16 has an electric power controller 32, which functions as a DC/DC converter. An input voltage from the battery 14 is step-upped by the electric power controller 32, and increased voltage is output to the LEDs 15A and 15B via an inductor 36 and a diode 34. The electric power controller 32 stabilizes an output voltage Vₒᵤₜ while monitoring a voltage at a connecting point 37, which is connected to a pin 32A of the controller 32, and therefore has the same voltage.

The LED 15B connects with the LED 15A in parallel with respect to the light-controller 16. Electric current i₁ and electric current i₂ flow through the LED 15A and the LED 15B, respectively. A resistor 39 having the resistor value R_{ref} connects with the LED 15A sequentially, and a variable resistor 38 having a variable resistor value RA connects with the LED 15B sequentially. An electric circuit branches from the connecting point 37 to the electric power controller 32.

Let a standard voltage for controlling an output voltage Vₒᵤₜ be designated by “V_{ref}”, the voltage across the LED 15A be designated by “VF1”, the voltage across the LED 15B be designated by “VF2”, and the voltage across the variable resistor 38 be designated by “VRA”. Then, the following equation is satisfied:
Vₒᵤₜ=VRA+VF2=V_{ref}+VF1 (1)
Therefore, the electric currents i₁ and i₂ satisfy the following equation:
Vₒᵤₜ=i₂×RA+VF2=i₁×R_{ref}+VF1 (2)
Since the values of the voltages VF1 and VF2 are substantially equal to each other, the difference between the electric current i₁ and the electric current i₂ is adjusted by changing the resistor value RA of the variable resistor 38. Thus, by lowering the resistor value RA relative to the resistor value R_{ref} of the resistor 39, the light-intensity of the LED 15A increases. The resistor value RA is set by operating the color balance button 17 shown in FIG. 1.

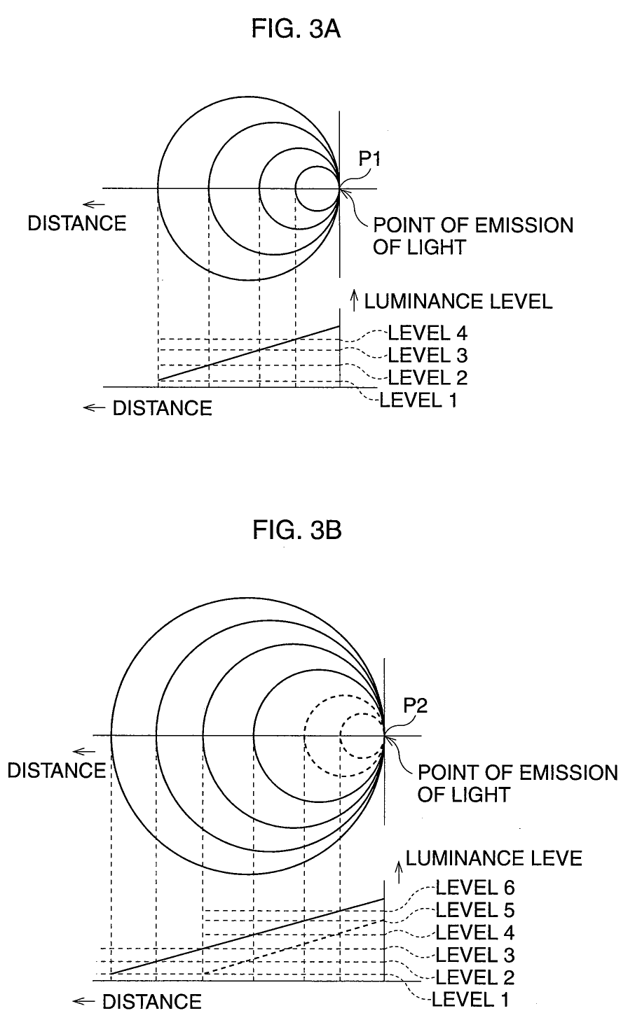

FIGS. 3A and 3B are views showing luminance distributions of the LEDs 15A and 15B.

In FIG. 3A, a luminance distribution of the LED 15A is represented by luminance contours, which represent different luminance levels. The luminance level becomes weaker as the distance from the tip portion 10A (the point of emission of light P1) becomes farther away. The luminance level 1 herein represents a minimum level necessary for observation.

In FIG. 3B, a luminance distribution of the LED 15B is shown. The luminance distribution of the LED 15B, in other words, the light-intensity of the LED 15B is larger than that of the LED 15A. Consequently, the total luminance distribution based on the two LEDs 15A and 15B becomes asymmetrical and unbalanced. Herein, the resistor value RA is set such that the ratio of the luminance of the LED 15A and the luminance of the LED 15B is set to 1:2.

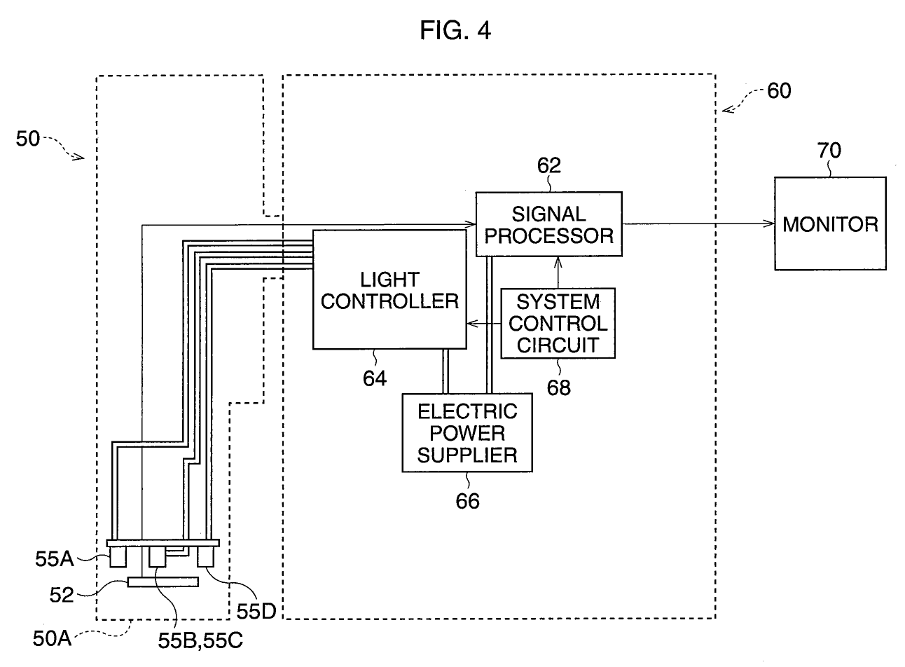

With reference to FIGS. 4 to 6, the second embodiment is explained. In the second embodiment, LEDs having different directivities of light are provided.

FIG. 4 is a block diagram of an electronic endoscope according to a second embodiment.

The electronic endoscope has a video-scope 50 and a video-processor 60, and a monitor 70 is connected to the video-processor 60. Four LEDs 55A, 55B, 55C, and 55D are provided at the tip portion 50A of the video-scope 50, and are controlled by a light-controller 64 in the video-processor 60. A system control circuit 68 controls a signal processor 62 and the light-controller 64. A power supplier 66 supplies electric power to each circuit. Light irradiated from the LEDs 55A to 55D is reflected off an observed portion, and an object image is formed on a CCD 52. Image-pixel signals corresponding to the object image are read from the CCD 52 and are fed to the signal processor 62, in which the image-pixel signals are subjected to various processes to generate video signals. The generated video signals are output to the monitor 70 so that the object image is displayed on the monitor 70.

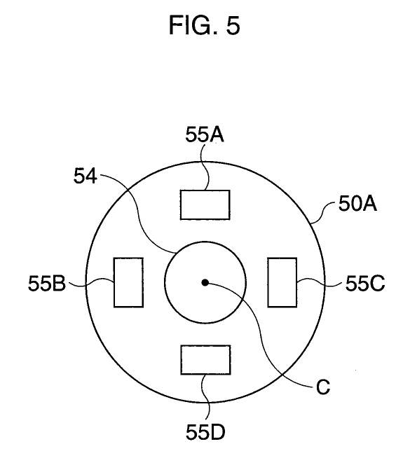

FIG. 5 is a view showing a front view of the tip portion 50A.

The plate-shaped LEDs 55A to 55D are arranged so as to be symmetrical with respect to the center axis C of the tip portion 50A, and are arranged around an objective lens 54 at regular intervals. Each of the LEDs 55A to 55D is a white LED that emits white light toward a frontal direction of the tip portion 50A. Each of the LEDs 55A to 55D has a diode element (not shown), and a transparent resin lens (not shown) which covers the diode element. The resin lens is composed of light-diffusion material, and functions as a lens having a focal length.

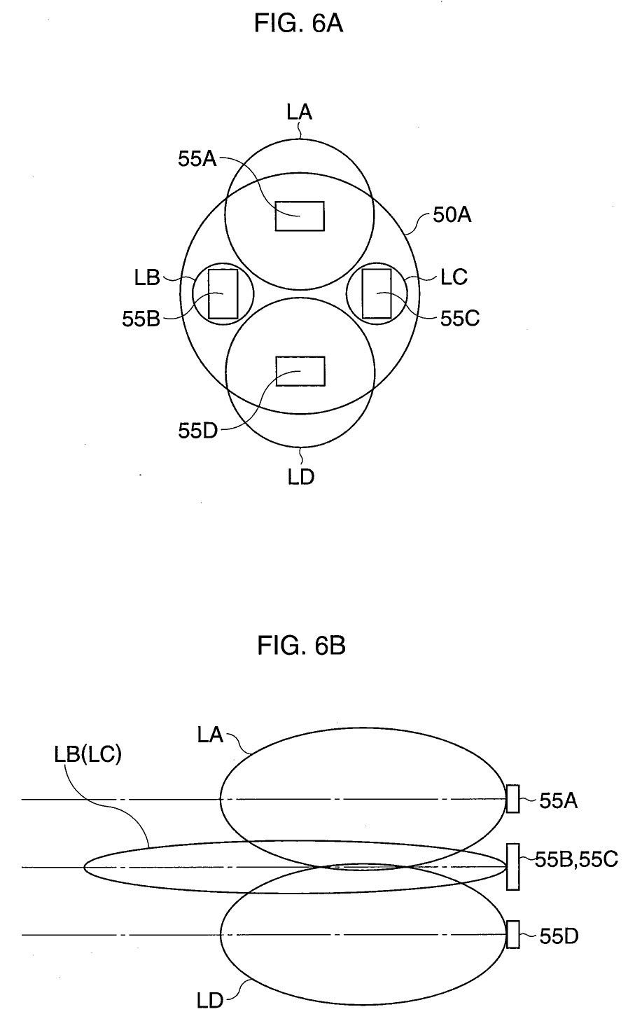

FIGS. 6A and 6B are views showing the directivities of the LEDs 55A to 55D.

In FIG. 6A, the directivity of light seen from the front view is shown. In FIG. 6B, the directivity of light seen from a side view is shown. Curved lines LA, LB, LC, and LD represent directivities of the LEDs 55A, 55B, 55C, and 55D, respectively. The directivity of the LED 55A, in other words, the range of irradiation of LED 55A, is the same as that of the LED 55D, opposite the LED 55A. The directivity of the LED 55B is the same as that of the LED 55C, opposite the LED 55B. Each of directivities depends upon the diffusion characteristics of the corresponding loaded resin.

As can be seen from FIG. 6B, the directivity of light of the LEDs 55A and 55D has the property that the expansions of their irradiated light are broad, and the irradiated light reaches only a close distance (see the curved lines LA and LD). On the other hand, the directivity of light of the LEDs 15B and 15C has the property that the expansion of their irradiated light is narrow, and the irradiated light reaches relatively far away (see the curved lines LB and LC). Consequently, the total luminance distribution based on the LEDs 15A to 15D becomes asymmetrical and unbalanced.

Optionally, an image fiber composed of a glass fiber may be used instead of the plastic image-fiber 12. The directivity of light may be changed by changing the focal length of the loaded resin. Optionally, a shell-shaped LED, or a chip-shaped LED may be used instead of the plate-like LED. Optionally, the LEDs may be provided in the processor. In this case, a light-guide composed of a fiber-optic bundle may be used.

Finally, it will be understood by those skilled in the arts that the foregoing description is of preferred embodiments of the device, and that various changes and modifications may be made to the present invention without departing from the spirit and scope thereof.

The present disclosure relates to subject matter contained in Japanese Patent Application No. 2005-231748 (filed on Aug. 10, 2005), which is expressly incorporated herein, by reference, in its entirety.

1. An endoscope comprising:
a plurality of LEDs (Light-Emitting Diodes) that illuminate an object and are disposed at a tip portion of said endoscope; and
a light-controller that controls said plurality of LEDs so as to cause a luminance difference among said plurality of LEDs.

2. The endoscope of claim 1, further comprising a variable resistor that connects with one LED among said plurality of LEDs, said plurality of LEDs connecting with one another in parallel with respect to said light-controller.

3. The endoscope of claim 2, further comprising a resistor value setter that sets a value of said variable resistor.

4. An endoscope comprising:
a plurality of LEDs (Light-Emitting Diodes) that illuminate an object and are disposed at a tip portion of said endoscope, said plurality of LEDs comprising LEDs that have different directivities of light; and
a light-controller that controls an emission of said plurality of LEDs.

5. The endoscope of claim 4, wherein said plurality of LEDs comprises at least one first LED in which the directivity is relatively broad, and at least one second LED in which the directivity is relatively narrow.

6. The endoscope of claim 4, wherein each of said plurality of LEDs comprises a resin that diffuses irradiated light and has a diffusion characteristic defining a directivity of light.

7. The endoscope of claim 4, wherein each of said plurality of LEDs comprises a lens that has a focal length defining a directivity of light.
